(19) United States
(12) Patent Application Publication
(10) Pub. No.: US 2011/0071198 A1
(43) Pub. Date: Mar. 24, 2011

(54) NICOTINIC ACID RECEPTOR LIGANDS

(76) Inventors: Robert J. Lefkowitz, Durham, NC (US); Scott M. Dewire, Durham, NC (US); Erin J. Whalen, Durham, NC (US); Jonathan D. Violin, Durham, NC (US); Robert W. Walters, Durham, NC (US)

(21) Appl. No.: 12/736,966

(22) PCT Filed: May 27, 2009
(86) PCT No.: PCT/US09/03218

§ 371 Date: Nov. 26, 2010

Related U.S. Application Data
(60) Provisional application No. 61/071,935, filed on May 27, 2008.

(51) Int. Cl. A61K 31/455; G01N 33/53; A61P 9/10; A61P 3/06

(52) U.S. Cl. 514/356; 436/501; 435/7.2

(57) ABSTRACT

The present invention relates, in general, to nicotinic acid receptor ligands and, in particular, to ligands that have a relative efficacy for activating a G-protein-coupled receptor function (e.g., signaling) that is greater than their relative efficacy for stimulating β-arrestin function (e.g., recruitment and/or signaling). The invention further relates to the use of such “biased ligands” to decrease triglycerides levels and to potentially increase high density lipoprotein (HDL) levels in patients and potentially lower low density lipoprotein (LDL) and/or very low density lipoprotein (VLDL) levels. In addition, the invention relates to methods of identifying such “biased ligands”.

This application claims priority from U.S. Provisional Application No. 61/071,935, filed May 27, 2008, the entire content of which is incorporated herein by reference.

This invention was made with government support under Grant Nos. 2R01HL016037-33 and 5R01HL070631-04 awarded by the National Institutes of Health. The government has certain rights in the invention.

### TECHNICAL FIELD

The present invention relates, in general, to nicotinic acid receptor ligands and, in particular, to ligands that have a relative efficacy for activating a G-protein-coupled receptor function (e.g., signaling) that is greater than their relative efficacy for stimulating β-arrestin function (e.g., recruitment and/or signaling). The invention further relates to the use of such “biased ligands” to decrease triglycerides in patients and to potentially increase high density lipoprotein (HDL) and potentially lower low density lipoprotein (LDL) and/or very low density lipoprotein (VLDL) levels. In addition, the invention relates to methods of identifying such “biased ligands”.

### BACKGROUND

Nicotinic acid, also known as niacin or vitamin B-3, has long been known to affect lipid profiles in humans. The pleiotropic effects of nicotinic acid therapy include the improvement of a number of cardiovascular risk factors. Specifically, nicotinic acid is the most effective high density lipoprotein (HDL) raising therapy currently known, and has also been shown to lower triglycerides and both very low density lipoprotein (VLDL) and low density lipoprotein (LDL) levels (Pike, Clin. Invest. 115(12):3400-3403 (2005)). Nicotinic acid therapy is associated with a significant side effect in which the recipient experiences a rather unpleasant cutaneous vasodilation or flushing response, which often includes a severe burning and itching sensation (Pike, Clin. Invest. 115(12):3400-3403 (2005)). This side effect occurs in ˜80% of patients and is often cited as the reason patients discontinue nicotinic acid therapy.

The nicotinic acid-induced flush results from activation of the 7-transmembrane G protein-coupled GPR109A receptor (Benyo et al, J Clin. Invest. 115(12):3634-3640 (2005)). In particular, nicotinic acid-mediated stimulation of GPR109A receptors expressed on Langerhans' cells in the skin leads to the secretion of prostaglandin D2 and consequent cutaneous vasodilation and flushing (Morrow, J. Invest. Dermatol. 98(5):812-815 (1992), Benyo et al, Mol. Pharmacol. 70(6):1840-1849 (2006)). This flushing response can be somewhat abrogated by taking aspirin prior to nicotinic acid dosing (Andersson et al, Acta Pharmacol. Toxicol. (Copenh) 41(1):1-10 (1977), Eklund et al, Prostaglandins 17(6):821-830 (1979)). Different dosing strategies have also been explored to abrogate flushing, including sustained release nicotinic acid, which is associated with increased hepatotoxicity, and extended release nicotinic acid (Niaspan), which is associated with somewhat decreased flushing (Guyton, Expert Opin. Pharmacother. 5(6):1385-1398 (2004)). Further, Merck has recently reported reaching primary phase III goals for its HDL raising drug, Cordaptive, a combination of extended release nicotinic acid (Niaspan) and a prostaglandin D2 inhibitor (laropiprant). There are, therefore, multiple approaches currently being taken to try and overcome this flushing side effect.

Seven-transmembrane receptors are capable of all manner of signaling, including G protein dependent and independent processes (Leflcowitz et al, Mol. Cell 24(5):643-652 (2006), DeWire et al, Alum. Rev. Physiol. 69:483-510 (2007), Violin and Lefkowitz, Trends Pharmacol. Sci. 28(8):416-422 (2007)). Recently, it has been appreciated that these distinct signaling processes may differentially contribute to the desired (therapeutic), as well as undesired (side-effects), traits of modern pharmaceuticals. That is to say, specific signaling pathways, such as those involving G proteins vs. those involving the β-arrestins, can transduce distinct signaling with distinct functional consequences. This raises the possibility that signaling from the receptor can be promoted with a bias for either the G protein or β-arrestin associated pathway. Moreover, this biased signaling at the receptor level can be directed by a specific ligand. This idea of “biased ligands” departs from the traditional view of receptor ligands as full agonists, partial agonists or antagonists, and opens up a much more dynamic drug-able space that includes ligands that are full agonists, partial agonists or antagonists independently for G protein activation and/or β-arrestins.

The present invention provides, at least in part, methods of identifying agents that can be used to decrease the level of triglycerides in a patient, and potentially decrease the level of LDL and/or VLDL and increase HDL, while avoiding or reducing flushing associated with administration of nicotinic acid.

### SUMMARY OF THE INVENTION

The present invention relates generally to nicotinic acid receptor ligands. More specifically, the invention relates to nicotinic acid receptor ligands that have a relative efficacy for activating a G-protein-coupled receptor function (e.g., signaling) that is greater than their relative efficacy for stimulating β-arrestin function (e.g., recruitment and/or signaling). The invention also relates to a method of decreasing triglycerides in a patient and potentially lowering the LDL and/or VLDL level and increasing the HDL level using such “biased ligands”. The invention further relates to methods of identifying such “biased ligands”.

Objects and advantages of the present invention will be clear from the description that follows.

### BRIEF DESCRIPTION OF THE DRAWINGS

FIGS. 1A and 1B. Nicotinic acid—induced decrease in cAMP, and increase in β-arrestin membrane recruitment in GPR109A-expressing HEK-293 cells. Cells also expressing the ICUE2 biosensor were treated with forskolin and increasing concentrations of nicotinic acid (NA). (FIG. 1A) Nicotinic acid (open circles) decreased cAMP in a dose-dependent fashion, and this response was inhibited by pertussis toxin (PTX; filled circles). Data are mean±SEM of 3 independent experiments. CFP, cyan fluorescent protein; FRET, fluorescence resonance energy transfer. (FIG. 1B) Cells were transfected with either β-arrestin1-mYFP or β-arrestin2-mYFP. Both β-arrestin isoforms resided in the cytosol prior to nicotinic acid stimulation (control; Con), and translocated to bind GPR109A in the membrane in response to treatment with 10 μM nicotinic acid. No translocation was noted in cells lacking GPR109A (not shown). Images are representative of 4 independent experiments. Original magnification, ×100.

FIGS. 2A-2C. Adipocytes, macrophages, and Langerhans cells express β-arrestins, and β-arrestin1 is recruited to the cell membrane with stimulation of GPR109A in Langerhans cells. (FIG. 2A) Cell lysates from differentiated 3T3-L1 adipocytes, differentiated THP-1 macrophages, and Langerhans cells (LHC) expressed both β-arrestin1 (Barr1) and β-arrestin2 (Barr2). (FIG. 2B) After stimulation with 10 μM nicotinic acid for 10 minutes, Langerhans cells were harvested, and membranes were separated from the cytosol, as demonstrated by presence of tubulin only in the cytosolic fractions. Increased β-arrestin1 was detected in the membranes after nicotinic acid stimulation, in contrast to control-treated samples. (FIG. 2C) Recruitment of β-arrestin1 to the membrane after nicotinic acid stimulation. *P=0.0014 versus nicotinic acid. Data are mean±SEM of 3 independent experiments.

FIGS. 3A and 3B. Conformational change in β-arrestin2 upon activation of GPR109A. (FIG. 3A) Dose dependency of the conformational changes in β-arrestin2 upon stimulation of GPR109A by nicotinic acid. Filled squares, cells expressing Luc-β-arr-YFP and empty vector; open squares, cells expressing Luc-β-arr-YFP and GPR109A. (FIG. 3B) Kinetics of nicotinic acid—induced conformational change in β-arrestin. Data are mean±SEM of 4 independent experiments.

FIGS. 4A-4C. Nicotinic acid—stimulated phosphorylation of ERK. (FIG. 4A) GPR109A-expressing HEK-293 cells were stimulated with 200 μM nicotinic acid, and cell lysates were analyzed for phosphorylated ERK (pERK) at varying times. Agonist stimulated activation of ERK in the presence or absence of pertussis toxin. tERK, total ERK. *P=0.027 versus control. (FIG. 4B) Expression of β-arrestin decreased after siRNA treatment. (FIG. 4C) Agonist stimulated ERK activation in the presence of control, β-arrestin1, β-arrestin2, or β-arrestin1 and β-arrestin2 siRNA. Graph shows phosphorylation of ERK 10 minutes after stimulation. **P<0.05 versus control. Data are mean±SEM of 3-6 independent experiments.

FIGS. 5A-5D. Nicotinic acid—induced binding of β-arrestin to cPLA₂ and phosphorylated cPLA₂. (FIGS. 5A and 5B) GPR109A-expressing HEK-293 cells were stimulated with 10 μM nicotinic acid or control for 10 minutes. Nicotinic acid stimulation increased binding of β-arrestin to cPLA₂ (FIG. 5A) and phosphorylated cPLA₂ (p-cPLA₂) (FIG. 5B). Arrow indicates phosphorylated cPLA₂ band. Equivalent amounts of cPLA₂ were present in each whole cell lysate (WCL). Equal amounts of β-arrestin were immunoprecipitated in control and nicotinic acid—treated samples. Moreover, β-arrestin was not immunoprecipitated with preimmune serum (not shown). (FIGS. 5C and 5D) Binding of β-arrestin to cPLA₂ (FIG. 5C) and phosphorylated cPLA₂ (FIG. 5D). *P=0.0075, **P=0.015 versus control. Data are mean±SEM of 5 independent experiments.

FIGS. 6A-6D. Role of β-arrestin1 in binding and activation of cPLA₂. (FIG. 6A) GPR109A-expressing HEK-293 cells were transfected with FLAG-β-arrestin1 or FLAG-β-arrestin2. Nicotinic acid stimulation increased binding of cPLA₂ to FLAG-β-arrestin1, but not FLAG-β-arrestin2. (FIG. 6B) Equivalent amounts of cPLA₂ and FLAG-β-arrestins were present in each whole cell lysate. Equal amounts of FLAG-β-arrestin were immunoprecipitated in control and nicotinic acid—treated samples. GPR109A-expressing HEK-293 cells were stimulated with 200 μM nicotinic acid, and cell lysates were analyzed for phosphorylated cPLA₂ at varying times. Agonist-stimulated activation of cPLA₂ in the presence of control siRNA, β-arrestin1 siRNA, or control siRNA plus either pertussis toxin or PD98059 (PD). (FIG. 6C) Binding of FLAG-β-arrestin to cPLA₂. *P=0.0004 versus respective control. (FIG. 6D) Activation or phosphorylation of cPLA₂ in siRNA-treated cells. **P=0.0085 versus respective 10-minute value; ***P=0.0047 versus respective 0-minute value. Data are mean±SEM of 3 independent experiments.

FIG. 7. Nicotinic acid induces antilipolysis in wild-type and β-arrestin-deficient mice. Nicotinic acid decreased FFA levels in wild-type C57BL/6 mice as well as mice deficient in β-arrestin1 or β-arrestin2. Nonesterified FFAs were measured in mice given i.p. injections of either vehicle alone or nicotinic acid at a dose of 10, 50, or 100 mg/kg. FFA levels are expressed as a percent of vehicle-treated control animals for each genotype. *P<0.0001 comparing the interaction of dose. The change in FFAs after nicotinic acid stimulation was not significantly different between wild-type, β-arrestin1-deficient, and β-arrestin2-deficient mice. Data are mean±SEM in control or nicotinic acid—treated animals (n=4−10 per condition).

FIGS. 8A-8E. Nicotinic acid—induced cutaneous flushing and activity of cPLA2 is attenuated in β-arrestin1-deficient mice. (FIG. 8A) Perfusion of the ventral ear in wildtype, β-arrestin1-deficient, or β-arrestin2-deficient mice was measured with laser Doppler. Baseline perfusion was measured for 150 seconds, then mice were given i.p. injections of 100 mg/kg nicotinic acid. Data are mean±SEM for change in perfusion as a function of time. (FIG. 8B) Total response to nicotinic acid, plotted as mean±SEM area under the curve. (FIG. 8C) Maximum response to nicotinic acid, plotted as mean±SEM. *P<0.0001 versus wild type. (FIG. 8D) Maximum response to PGD₂, plotted as mean±SEM (n=15−25 per condition). (FIG. 8E) Nicotinic acid—stimulated release of eicosanoids was measured in peritoneal macrophages. Thioglycollate-elicited peritoneal macrophages were loaded with H3-arachidonic acid (H3-AA) for 24 hours, and then rinsed to remove arachidonic acid not incorporated into cell membrane lipids. Macrophages were stimulated for 10 minutes with 200 μM nicotinic acid, and radioactivity released into the media was measured. Data are mean±SEM (n=4 per condition) for change in radioactivity, plotted as a percentage of the maximum response. **P=0.0001 versus nicotinic acid—treated wild type.

FIGS. 9A and 9B. MK-0345-induced G protein signaling, β-arrestin conformational changes, and recruitment. (FIG. 9A) Cells expressing GPR109A and the ICUE2 biosensor were treated with forskolin and MK-0354 (MK). MK-0354 (open circles) decreased cAMP in a dose-dependent fashion, and this response was inhibited by pertussis toxin (filled circles). (FIG. 9B) Cells expressing GPR109A and the BRET reporter Luc-β-arr-YFP were treated with nicotinic acid (open squares) or MK-0354 (open circles). MK-0354 failed to induce conformational changes in β-arrestin2. Data are mean±SEM of 3 independent experiments. (FIG. 9C) Cells expressing GPR109A β-arrestin1-mYFP were treated with nicotinic acid, MK-0354, or both. Prior to nicotinic acid stimulation, β-arrestin1 resided in the cytosol; it translocated to bind GPR109A in the membrane in response to 10 μM nicotinic acid. No translocation was noted in cells stimulated with 200 μM MK-0354 or in cells treated with 10 μM nicotinic acid in the presence of 200 μM MK-0354. Images are representative of 4 independent experiments. Original magnification, ×100.

FIG. 10. Mice exhibit rapid tachyphylaxis in the flushing response to increasing doses of nicotinic acid. Perfusion of the ventral ear in age and weight matched wild-type C57B1/6 mice was measured with laser doppler. Baseline perfusion was measured for 150 seconds then mice were given intraperitoneal injections of nicotinic acid at times indicated by the arrows. The first dose was 25 mg/kg, the second dose was 50 mg/kg and the third dose was 100 mg/kg. Wild-type (rechallenge) mice received all three doses of nicotinic acid and wild-type (control) mice received the first dose of nicotinic acid followed by two subsequent doses with vehicle alone. Plotted values represent the mean change in perfusion as a function of time from 8 animals.

### DETAILED DESCRIPTION OF THE INVENTION

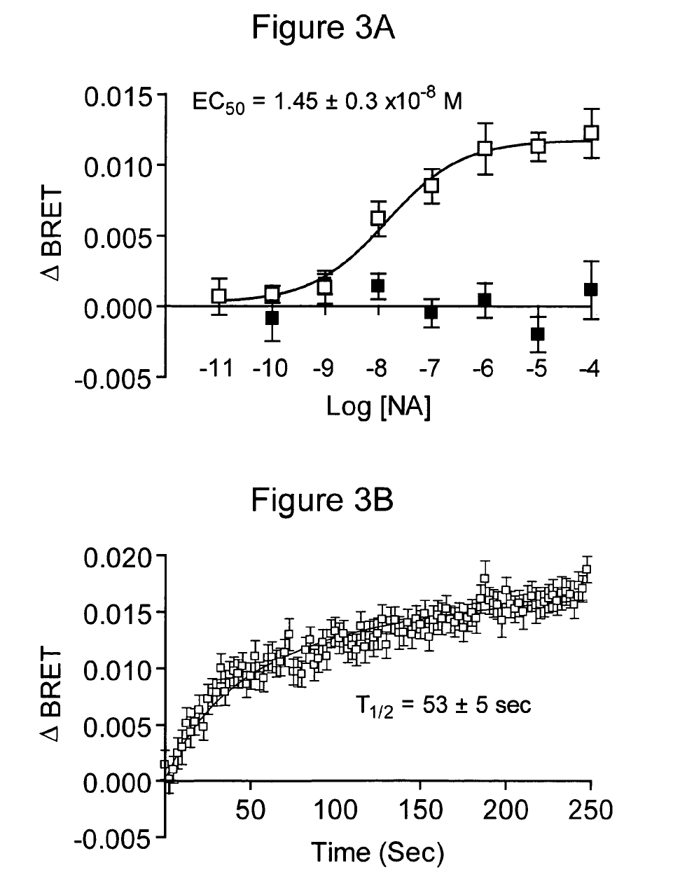

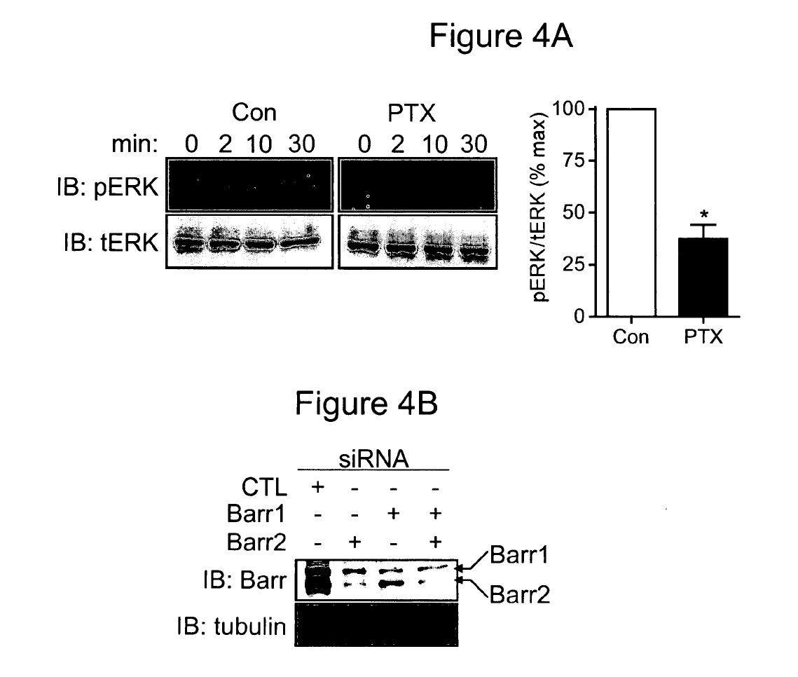

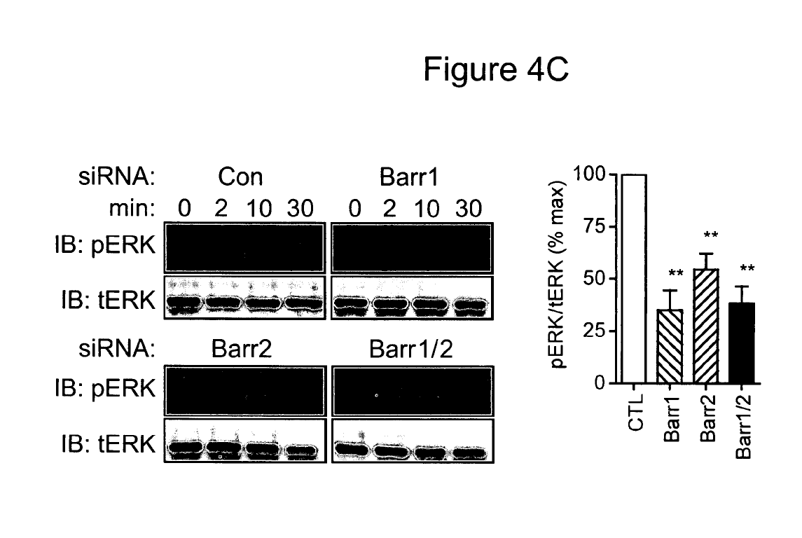

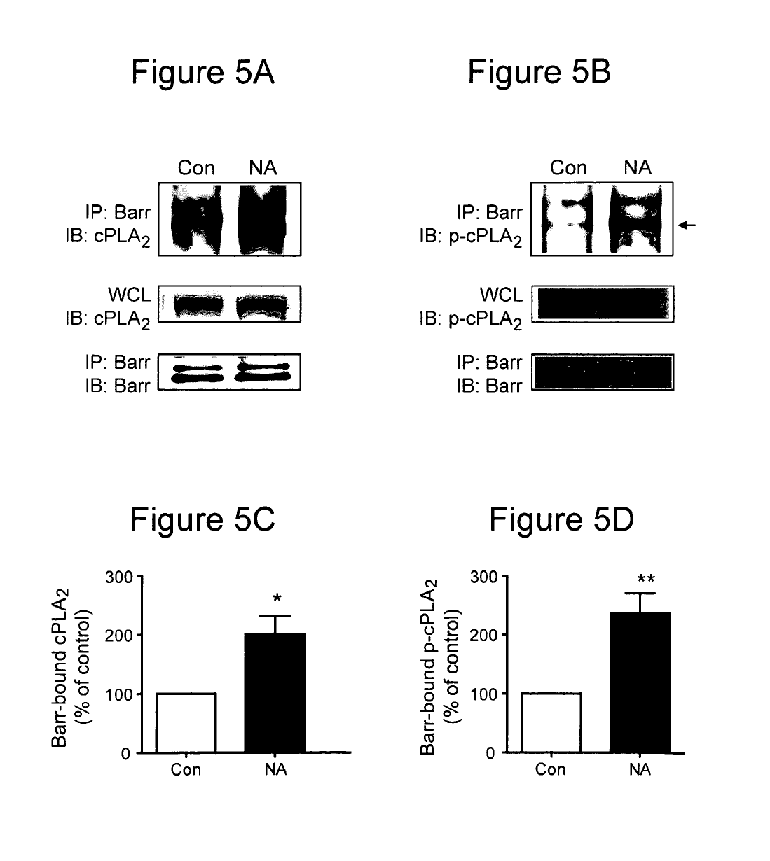

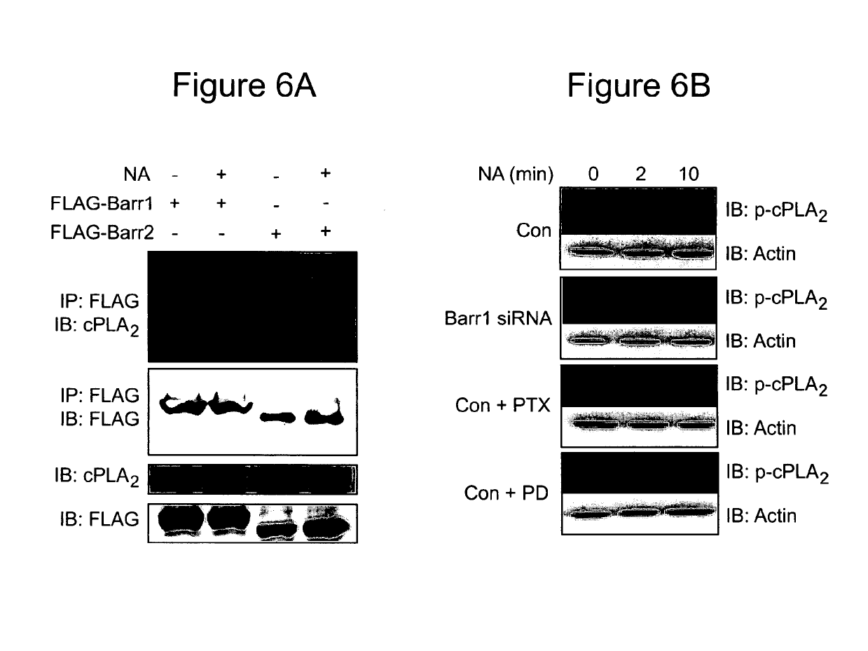

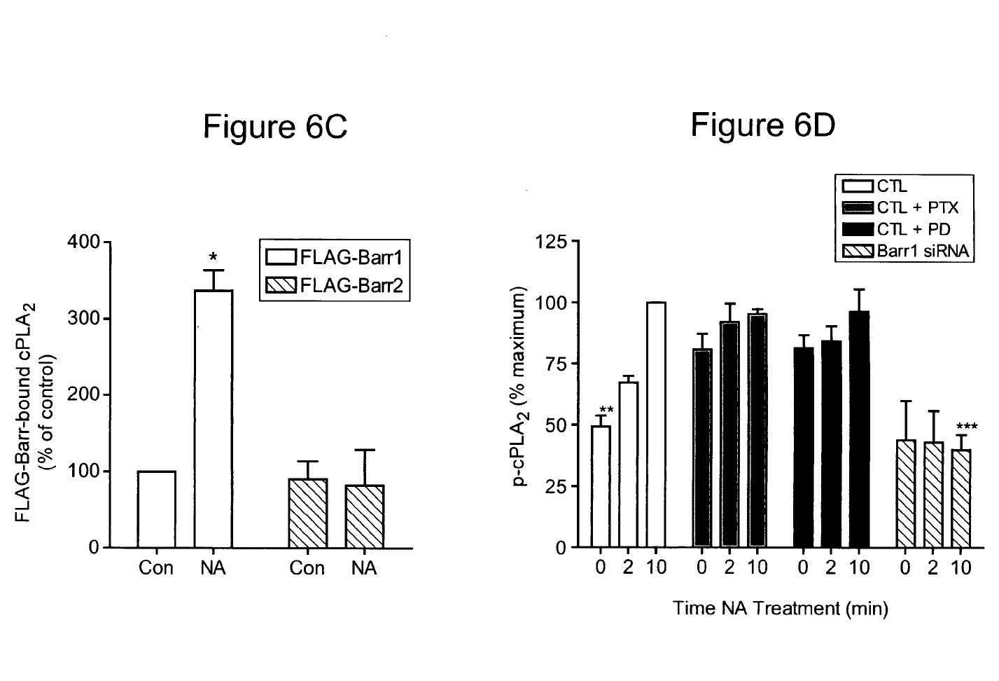

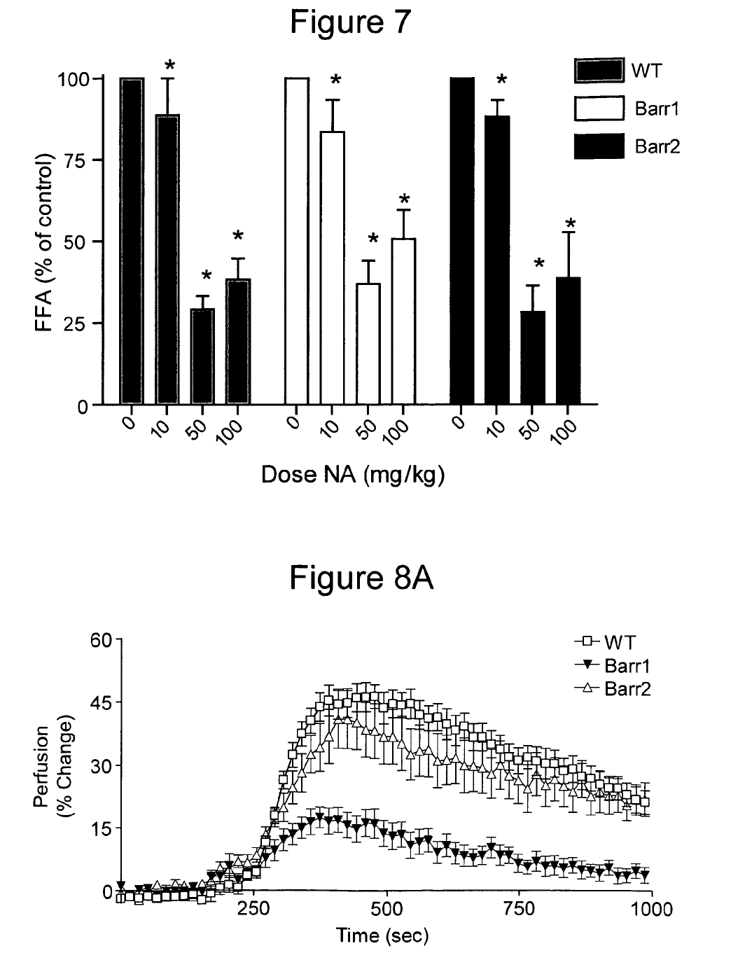

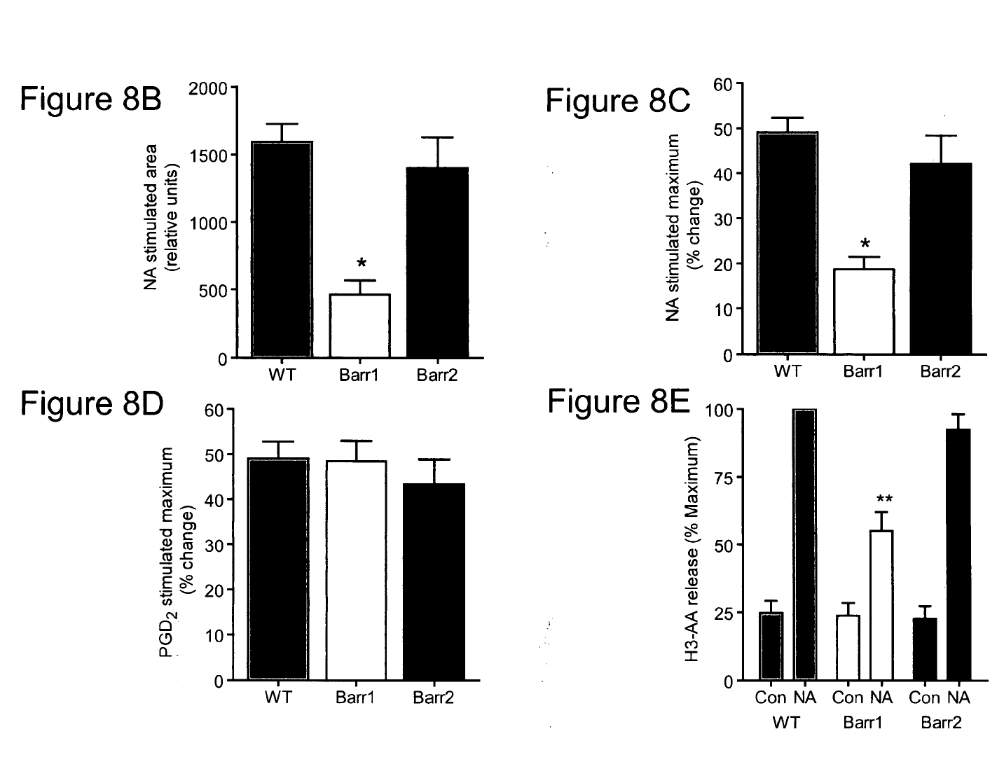

Nicotinic acid-induced flushing results from activation of the GPR109A receptor (Benyo et al, J. Clin. Invest. 115(12):3634-3640 (2005)). This receptor couples to the heterotrimeric G protein, Gᵢ (Sakai et al, Arterioscler. Thromb. Vasc. Biol. 21(11):1783-1789 (2001), Richman et al, J. Biol. Chem. 282(25):18028-18036 (2007)). It is shown in the Examples that follow that agonist (nicotinic acid)-induced stimulation leads to: Pertussis toxin sensitive lowering of cAMP (FIG. 3); recruitment of β-arrestins to the cell membrane (FIGS. 3 and 4); activating conformational change in β-arrestin (FIG. 5); β-arrestin dependent signaling to ERK MAP kinases (FIG. 6); binding of β-arrestin1 to activated cytosolic phospholipase A₂ (FIG. 7); and β-arrestin1 dependent activation of cytosolic phospholipase A₂ and release of arachidonate (FIG. 8), the precursor of prostaglandin D₂, the vasodilator responsible for the flushing response to nicotinic acid. Furthermore, the adverse side effect of cutaneous vasodilation/flushing associated with nicotinic acid therapy is mediated by β-arrestin1.

The present invention is based, at least in part, on the realization that the therapeutically beneficial effects of nicotinic acid can be mechanistically dissociated from its adverse side effects. In accordance with the invention, GPR109A agonists that are biased against β-arrestin1 activation can be used as therapeutic modalities for lowering triglycerides and potentially raising HDL and lowering LDL and/or VLDL with significantly reduced cutaneous vasodilation/flushing relative to, for example, nicotinic acid. These “biased ligands” can be used alone or in combination with other lipid modulating drugs, such as statins or other HMG-CoA reductase inhibitors.

In one embodiment, the invention relates to methods of identifying GPR109A ligands biased against β-arrestin1. A combined screening approach can be used in which the ability of a given ligand to activate G proteins (e.g., GPR109A) is compared to the ability of that ligand to stimulate recruitment of β-arrestins (e.g., (β-arrestin1) and/or β-arrestin mediated signaling. The results of the two screens can be plotted on opposing axes and a line of unity drawn. Typically, 7 transmembrane receptor (7TMR) agonists are equally efficacious for both G protein and β-arrestin signaling, that is, when the results of the two screens for a series of ligands are plotted, they typically show a linear relationship (see graph below). The relative efficacies of “biased ligands” of the instant invention can be readily appreciated from the graph below where the vertical axis is a measure of β-arrestin/GRK-dependent signaling (e.g., recruitment of β-arrestin to the receptor as assayed, for example, by FRET (see Examples below)) and the horizontal axis is a measure of G-protein dependent signaling (e.g., inhibition of cAMP generation (Gᵢ)). 7TMR ligands that fall on the diagonal line are “unbiased” in that, upon binding to the receptor, their relative effects on β-arrestin/GRK-dependent signaling and G-protein dependent signaling functions are essentially equivalent. The “biased ligands” of the instant invention, upon binding to the receptor, have a greater positive effect on G-protein dependent signaling function than on β-arrestin/GRK-dependent function and thus fall below and/or to the right of the line and in the shaded portion of the graph (biased ligands of the invention include agonists of G-protein dependent signaling, that is, ligands with positive efficacy for G-protein signaling falling to the right of the vertical axis and below the horizontal):

The bias of a particular ligand can be expressed using the following equation (1):

Bias=[e^{-(G protein activity)}][e^{(β-arrestin function)}]

where:
e=base of the natural logarithm,
G protein activity=percentage of a reference agonist function, and
β-arrestin function=percentage of a reference agonist function.

In accordance with the invention, the reference agonist can be a known ligand for GPR109A (e.g., nicotinic acid).

In determining bias of a ligand (or candidate ligand (e.g., a test compound)), G protein activity mediated by GPR109A can be measured using any of a wide variety of assays, including those well known in the art. For example, G protein activity can be assayed by determining the level of calcium, cAMP, diacylglycerol, or inositol triphosphate in the presence and absence of the ligand (or candidate ligand). G protein activity can also be assayed, for example, by determining phosphatidylinositol turnover, GTP-γ-S loading, adenylate cyclase activity, GTP hydrolysis, etc. in the presence and absence of the ligand (or candidate ligand). (See, for example, Kostenis, Curr. Pharm. Res. 12(14):1703-1715 (2006).)

Similarly, β-arrestin function mediated by a GPR109A in response to a ligand (or candidate ligand (e.g., a test compound)) can be measured using any of a variety of assays. For example, β-arrestin recruitment to the GPR109A or GPR109A internalization can be assayed in the presence and absence of the ligand (or candidate ligand). Advantageously, the β-arrestin function in the presence and absence of a ligand (or candidate ligand) is measured using resonance energy transfer (e.g., FRET), bimolecular fluorescence (e.g., BRET), enzyme complementation, visual translocation, co-immunoprecipitation, cell fractionation or interaction of β-arrestin with a naturally occurring binding partner. (See, for example, Violin et al, Trends Pharmacol. Sci. 28(8):416-427 (2007); Carter et al, J. Pharm. Exp. Ther. 2:839-848 (2005); PCT/US2007/018394 filed Aug. 20, 2007; and PCT/US2008/002257 filed Feb. 21, 2008.)

One skilled in the art will appreciate that GRK activity can be used as a surrogate for β-arrestin function. β-arrestin function mediated by a GPCR in response to a ligand (or candidate ligand) can thus be reflected by changes in GRK activity, as evidenced by changes in receptor internalization or phosphorylation.

While the relative efficacies for G protein activity and β-arrestin functions for a given ligand (or candidate ligand) acting on a GPR109A are preferably determined by assays in eukaryotic cells (e.g., mammalian cells (e.g., human cells), insect cells, avian cells, or amphibian cells, advantageously, mammalian cells), one skilled in the art will appreciate from a reading of this disclosure that appropriate assays can also be performed in prokaryotic cells, reconstituted membranes, and using purified proteins in vitro. Examples of such assays include, but are not limited to, in vitro phosphorylation of purified receptor by GRKs, GTP-γ-S loading in purified membranes from cells or tissues, and in vitro binding of purified β-arrestins to purified receptors upon addition of ligand (or candidate ligand) (with or without GRKs present in the reaction). (See, for example, Pitcher et al, Science 257:1264-1267 (1992); Zamah et al, J. Biol. Chem. 277:31249-31256 (2002); Benovic et al, Proc. Natl. Acad. Sci. 84:8879-8882 (1987).)

The above equation (1) measures the distance from the diagonal line in the above-presented graph, and expresses that distance, for compounds with G protein activity and β-arrestin function ranging between 0 and 1, as a number between −0.63 and +0.63, where −0.63 is a perfectly G protein-biased ligand and +0.63 is a perfectly β-arrestin-biased ligand. This range will vary for “superagonists” with activity/function greater than 1, and/or “inverse agonists” with activity/function less than 0. Full agonists, antagonists, and partial agonists with equal efficacies for both pathways, have a value of zero (as discussed above (and further below), the number resulting from application of the equation above is relative to a reference agonist for GPR109A (e.g., nicotinic acid)). It will be appreciated that two ligands that differ significantly in their ability to stimulate each pathway (i.e., G protein activity and β arrestin function), yet lie the same distance off the line, will have the same value in the above equation.

By way of example, if G protein activity mediated by GPR109A is determined for reference agonist “X” as being 400 units (as measured by assay “A”) and the G protein activity mediated by that GPR109A is determined for ligand (or candidate ligand) “Y” as being 300 units (also as measured by assay “A”), the G protein activity of ligand (or candidate ligand) “Y” relative to reference agonist “X” is 300/400=0.75. If β-arrestin function mediated by the GPR109A for reference agonist “X” is 200 units (as measured by assay “B”) and the same β-arrestin function mediated by that GPR109A is determined for ligand (or candidate ligand) “Y” as being 50 units (also as measured by assay “B”), then ther β-arrestin function of ligand (or candidate ligand) “Y” relative to reference agonist “X” is 50/200=0.25. Using equation (1), the bias of ligand (or candidate ligand) “Y” is thus:

Bias=[e^{-0.25}]=0.47−0.78=−0.31.

The value derived using the above equation (1) for preferred biased ligands of the invention is →0.05, →0.075, →0.1, →0.2, →0.3, →0.4, or →0.5. Biased ligands having a bias value in the range of −0.05 to −1, −0.075 to −1, −0.1 to −1, −0.2 to −1, −0.3 to −1, −0.4 to −1, or −0.5 to −1 are preferred.

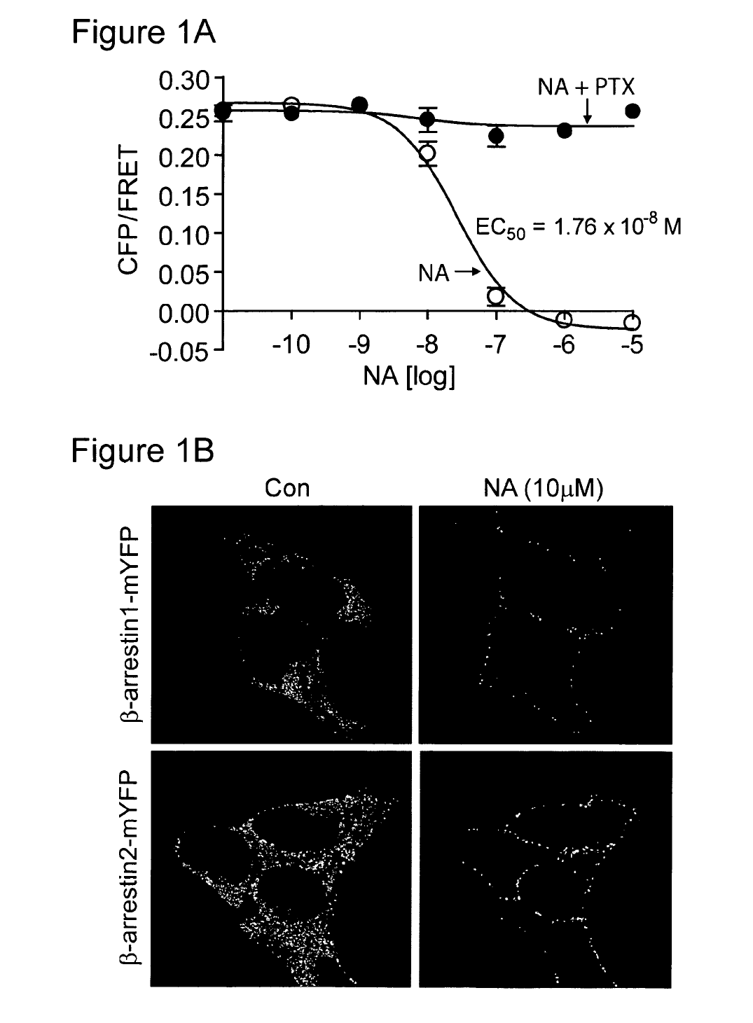

Even though the absence of β-arrestin1 reduces nicotinic acid-induced flushing (see Examples below), and a G-protein biased GPR109A ligand that does not engage β-arrestin1 is predicted to retain therapeutic benefits while exhibiting reduced flushing, some flushing response may still remain (see FIG. 1). The invention further relates to a therapeutic strategy that can reduce or eliminate any remaining flushing response.

In accordance with this aspect of the invention, a patient is given an initial low to moderate dose of a G protein biased GPR109A ligand (e.g., a biased ligand identifiable using the methods described above) closely followed by a much larger therapeutically beneficial dose to reduce or eliminate flushing associated with the biased ligand. This strategy can also be used to reduce flushing for unbiased GPR109A ligands, including nicotinic acid. For example, a low to moderate dose of nicotinic acid (e.g., 50-300 mgs) can be administered closely followed (e.g., approx. 30 min. later) by a much larger therapeutically beneficial dose (e.g., 1000-2000 mgs) of nicotinic acid. A GPR109A ligand, such as nicotinic acid, can be administered orally, for example, in pill or capsule form. A pill or capsule containing a low to moderate dose (e.g., 50-300 mgs for nicotinic acid) of the ligand available for immediate release can be combined with a delayed release (e.g., approx. 30 min.)larger therapeutically beneficial dose (e.g., 1000-2000 mgs for nicotinic acid). Such dosing strategies allow the patient to develop tolerance to the flushing response associated with the ligand, e.g., nicotinic acid (or derivative thereof) over a shorter period of time (hours to days), while still getting the larger dose of nicotinic acid (or derivative) required to obtain a therapeutic benefit. This strategy provides the patient with a single, shorter and less intense round of tolerance development.

As pointed out above, nicotinic acid-induced cutaneous vasodilation/flushing is the result of GPR109A receptor activation on Langerhans' cells in the skin and the consequent secretion of prostaglandin D2 (Morrow et al, J. Invest. Dermatol. 98(5):812-815 (1992), Benyo et al, Mol. Pharmacol. 70(6):1844-1849 (2006)). Nicotinic acid also inhibits lipolysis in adipocytes, decreasing serum free fatty acids and triglycerides, and this effect is mediated by GPR109A (Tunaru et al, Nat. Med. 9(3):352-355 (2003)). In addition to the triglyceride lowering effect, nicotinic acid also improves a number of other cardiovascular risk factors. Specifically, nicotinic acid is the most effective high density lipoprotein (HDL) raising therapy currently known, and has also been shown to lower very low density lipoprotein (VLDL) and low density lipoprotein (LDL) (Pike, Clin. Invest. 115(12):3400-3403 (2005)). While the free fatty acid and triglyceride lowering effects of nicotinic acid are clearly mediated by GPR109A, it is not yet known if all or only part of its beneficial effects on HDL, VLDL and LDL are also mediated by this receptor (Guyton, Curr. Opin. Lipidol. 18(4):415-420 (2007)). GPR109A receptors are found primarily in adipose tissue, the spleen, adrenal glands and lungs, and are all but absent from the liver and intestines, which are the main sites of HDL synthesis and metabolism (Tunaru et al, Nat. Med. 9(3):352-355 (2003), Wise et al, J. Biol. Chem. 278(11):9869-9874 (2003)). Thus, there may be additional mechanisms of action for the beneficial effects of nicotinic acid on lipoprotein profiles, mediated through sites other than GPR109A. For example, nicotinic acid at high concentrations has been shown to directly inhibit hepatic diacylglycerol acetyltransferase (Ganjii et al, J. Nutr. Biochem. 14(6):298-305 (2003), Jin et al, Arterioscler. Thromb. Vasc. Biol. 19(4):1051-1059 (1999)), thus inhibiting hepatic triglyceride synthesis, which increases apo B degradation, and consequently decreases VLDL and LDL production and secretion. Nicotinic acid has also been shown, via a yet to be identified mechanism, to inhibit the uptake and removal of HDL by the liver, resulting in increased circulating HDL levels (Jin et al, Arterioscler. Thromb. Vasc. Biol. 17(10):2020-2028 (1997)). Thus, multiple sites of action may be involved in the pleiotropic actions of nicotinic acid on lipoproteins beyond the clear effect on free fatty acids and triglycerides mediated through GPR109A.

Therefore, in a further embodiment, the invention relates to the use of a GPR109A antagonist to block nicotinic acid induced flushing, in combination or as a pre-treatment with nicotinic acid or a derivative thereof, for increasing HDL and decreasing VLDL and LDL in humans. Such an antagonist would be defined by its ability to block nicotinic acid-induced β-arrestin at the GPR109A. This approach can be used in combination with other lipid modulating therapies.

The invention further relates to compositions comprising at least one biased ligand formulated with an appropriate carrier. The composition can be in dosage unit form (e.g., a tablet or capsule). The composition can also be present, for example, as a solution (e.g., a sterile solution) or suspension, or as a gel, cream, ointment, aerosol or powder. Approaches suitable for delivering peptide and non-peptide biased ligands of the invention, including oral, transdermal, intrathecal, inhalation, IV, IP, IM, IN, delivery, are known in the art. (See, for example, Morishita et al, Drug Discovery Today 11:905-910 (2006), Ali et al, Letters in Peptide Science 8:289-294 (2002), and Hamman et al, Drug Target Insights 2:71-81 (2007), as well as the references cited in these reviews). Optimum formulations and dosing regimens can be determined by one skilled in the art and can vary with the biased ligand, the patient and the effect sought.

The present invention also relates to methods of identifying a biased ligand for a GPR109A. Such methods can comprise: i) determining the effect of a test compound on GPR109A-mediated G-protein activity, and ii) determining the effect of the test compound on GPR109A-mediated β-arrestin function, wherein a test compound that has a greater positive effect on GPR109A-mediated G-protein activity than on GPR109A-mediated β-arrestin function, relative to a reference agonist for both GPR109A-mediated G-protein activity and GPR109A-mediated β-arrestin function, is a biased ligand. Such methods can be used to identify a candidate therapeutic that can be used to modulate (e.g., inhibit) cutaneous vasodilation/flushing associated with nicotinic acid therapy. For example, candidate therapeutics can be identified by: i) determining the effect of a test compound on G-protein activity mediated by a GPR109A relevant to the physiological process, and ii) determining the effect of the test compound on β-arrestin function mediated by that GPR109A, wherein a test compound that has a greater positive effect on G-protein activity than on β-arrestin function mediated by GPR109A, relative to a reference agonist for both the G-protein activity and β-arrestin function mediated by the GPR109A, is such a candidate therapeutic.

One embodiment of this aspect of the invention comprises evaluating the relative efficacy of a test compound to stimulate G protein dependent pathways compared to its efficacy to stimulate β-arrestin/GRK function (e.g., association with the receptor or signaling), for example, to promote β-arrestin membrane translocation (the most proximal event in β-arrestin signaling). In a preferred approach, a fluorescence resonance energy transfer (FRET)-based assay is used to assess β-arrestin/GRK function stimulating efficacy. As described in PCT/US2007/018394, GRK/β-arrestin efficacy can be measured as the rate of β-arrestin recruitment to a receptor in response to ligand, where the receptor/β-arrestin interaction is measured by FRET or bioluminescent resonance energy transfer (BRET) (see also PCT/US2008/002257). For example, β₂AR-mCFP and β-arrestin-mYFP undergo FRET after addition of agonists with a quantifiable rate. This rate of FRET increase is a measure of ligand-stimulated GRK activity, which regulates β-arrestin function, and thus quantifies a ligand's β-arrestin/GRK efficacy. Details of a particularly preferred assay are provided in Example 5 of PCT/US2007/018394. This method can be adapted for use with a fluorescence plate reader for high-throughput screening of agonists and antagonists, which can thereby provide a rapid screen for β-arrestin/GRK biased ligands.

As noted above, and as described in PCT/US2007/018394, other assays that can be used to measure β-arrestin function include: receptor/β-arrestin co-immunoprecipitation, receptor/β-arrestin crosslinking, receptor/β-arrestin BRET, receptor/β-arrestin bimolecular fragmentation complementation, receptor/β-arrestin translocation imaging, receptor internalization, receptor phosphorylation, and β-arrestin associated phosphorylated ERK (Violin et al, Trends Pharmacol. Sci. 28(8):416-422 (2007)). As described above, approaches that can be used to measure G-protein mediated signaling function include assays for adenylate cyclase and/or cyclic AMP accumulation (ICUE (DiPilato et al, Proc. Natl. Acad. Sci. USA 101:16513 (2004)), radioimmunoassays, ELISAs, GTPase assays, GTPgammaS loading assays, intracellular calcium accumulation assays, phosphotidyl inositol hydrolysis assays, diacyl glycerol production assays (e.g., liquid chromatography, FRET based DAGR assay (Violin et al, J. Biol. Chem. 161:899 (2003)), receptor-G protein FRET assays, measures of receptor conformation change, receptor/G protein co-immunoprecipitation, ERK activation, phospholipase D activation, ion channel activation (including electrophysiologic methods), and cyclic GMP changes. (See, for example, Thomsen et al, Curr. Opin. Biotech. 16:655-665 (2005).) (See also PCT/US2008/002257.)

The therapeutic efficacy of the biased ligands of the invention, including those identifiable using the methods described above, can be increased using modifications known in the art to improve pharmacodynamic profile (e.g., increased affinity, etc), to prevent degradation (for peptides this can include N-acetylation and C-amidation, etc), to increase absorption, to allow for different routes of administration and different dosing strategies (including the addition of polyethylene glycol (PEGylation), lipids and protective salting, etc) and to modulate excretion. (See, for example, Morashita et al, Drug Discovery Today 11(19/20):905-910 (2006); Hamman et al, Drug Target Insights 2:71-81 (2007); Ali et al, Letter in Peptide Science 8:289-294 (2002); Whitfield et al, J. Bone

Certain aspects of the invention can be described in greater detail in the non-limiting Examples that follows. (See also Wei et al, Proc. Natl. Acad. Sci. USA 100:10782-10787 (2003); Wei et al, J. Biol. Chem. 279:48255-48261 (2004); Barnes et al, J. Biol. Chem. 280:8041-8050 (2005); Ren et al, Proc. Natl. Acad. Sci. USA 102:1448-1453 (2005); Kim et al, Proc. Natl. Acad. Sci. USA 102:1442-1447 (2005); Ahn et al, J. Biol. Chem. 297:7808-7811 (2004); Ahn et al, Proc. Natl. Acad. Sci. USA 100:1740-1744 (2003); Ahn et al, J. Biol. Chem. 279:35518-35525 (2004); Gesty-Palmer et al, J. Biol. Chem. 281:10856-10864 (2006); Hunton et al, Mol. Pharm. 67:1229-1236 (2005); Rajagopal et al, Circulation 112(17):U237-951 Suppl. 5 (2005); Rajagopal et al, J. Clin. Invest. 115(11):2971 (2005), Richman et al, J. Biol. Chem. 282(25):18028-18036 (2007)); Walters et al, J. Clin. Invest. 119(5):1312-1321 (2009), Epub 2009 Apr. 6.)

### EXAMPLE 1

### Experimental Details

Materials. Nicotinic acid, PGD₂, and (2-hydroxypropyl)-β-cyclodextrin were obtained from Sigma-Aldrich. MK-0354 was a gift from J. Richman (Arena Pharmaceuticals Inc., San Diego, Calif., USA). Pertussis toxin and the ERK inhibitor PD98059 were obtained from Calbiochem. Nicotinic acid [5,6-³H] was obtained from American Radiolabeled Chemicals. Arachidonic acid [5,6,8,9,12,14,15-³H(N)] was obtained from PerkinElmer. Coelenterazine h was purchased from Promega, and 96-well microplates for the BRET assay were purchased from Corning Inc.

Plasmids. β-arrestin1-mYFP, β-arrestin2-mYFP, FLAG-β-arrestin1, and FLAG-β-arrestin2 were produced as described previously (Violin et al, J. Biol. Chem. 281:20577-20588 (2006)). FLAG-GPR109A/pcDNA3.1 was a gift from S. Offermanns (University of Heidelberg, Heidelberg, Germany). The Luc-β-arr-YFP construct was provided by M. Bouvier (Université de Montréal, Montréal, Quebec, Canada).

Cell culture. 3T3-L1 cells were maintained in Dulbecco modified Eagle medium supplemented with 10% calf serum and 1% penicillin/streptomycin solution (Sigma-Aldrich), and the cells were differentiated by allowing them to reach confluence. THP-1 cells were maintained in RPMI-1640 medium supplemented with 10% fetal bovine serum, 1% penicillin/streptomycin solution, 1 mM sodium pyruvate, 10 mM HEPES, 4.5 g/l glucose, 1.5 g/l bicarbonate, and 0.05 mM 2-mercaptoethanol and were differentiated as previously described (Meyers et al, Atheroscloersis 192:253-258 (2007)). Langerhans cells were purchased and maintained according to the manufacturer's instructions (MatTek Corp.). HEK-293 cells were maintained in modified Eagle medium supplemented with 10% fetal bovine serum and 1% penicillin/streptomycin solution (Sigma-Aldrich). Cells were transfected with FuGENE 6 (Roche Applied Science). All transfections used 3 μg plasmid in a 10-cm tissue culture plate. Cells expressing GPR109A alone were selected with 400 μg/ml G418 (Sigma-Aldrich), and colonies of stable transfectants were isolated. Cells expressing GPR109A in combination with the ICUE2 biosensor were selected with 400 μg/ml G418 and 300 μg/ml Zeocin (Invitrogen).

ICUE cAMP assay. HEK-293 cells stably overexpressing both FLAG-GPR109A and the cAMP biosensor ICUE2 were stimulated with nicotinic acid or MK-0354 for 3 minutes, followed by stimulation with 10 μM forskolin for 4 minutes. Intracellular cAMP concentrations were measured as a fluorescence resonance energy transfer (FRET) ratio as follows: cyan fluorescent protein (CFP) intensity (438/32 emission bandpass filters; Semrock) relative to FRET intensity (542/27 emission filter (DiPilato et al, Proc. Natl. Acad. Sci. USA 101:16513-16518 (2004), Violin et al, J. Biol. Chem. 283:2949-2961 (2008))). Experiments were performed on a NOVOstar plate reader (BMG Labtech).

β-Arrestin translocation assays. HEK-293 cells stably expressing FLAG-GPR109A were transiently transfected with β-arrestin1-mYFP or β-arrestin2-mYFP using FuGENE 6 (Roche Applied Science). Cells were treated with nicotinic acid or MK-0354, and images were taken at 5-minute intervals after stimulation. Differentiated THP-1 cells were serum starved for 6 hours and subsequently stimulated with 200 μM nicotinic acid for 10 minutes. The plates were transferred on ice, and cells were washed twice with ice-cold PBS and then scraped in PBS containing complete protease inhibitor cocktail (Roche Applied Sciences). Cells were lysed by brief sonication and then centrifuged at 3,000 g for 5 minutes to remove unbroken cells and nuclear fractions. Subsequently, the supernatant was centrifuged at 21,000 g for 30 minutes to separate the membrane fraction (in the pellet) and cytosolic fraction (in the supernatant). The membrane pellet was resuspended in PBS, proteins in the membrane and the cytosolic fractions were measured by Bradford assay, and β-arrestins were detected by Western blot analysis.

BRET assay. BRET assays were performed as described previously (Charest et al, EMBO Rep. 6:334-340 (2005)). Briefly, at 24 hours after transfection, HEK-293 cells coexpressing the receptor and the biosensoi were distributed in fibronectin-coated 96-well microplates (white wall, clear bottom). Before the assay, cells were washed twice with PBS, the transparent bottom of the plate was covered with a white back-tape adhesive, and cells were incubated with coelenterazine h (final concentration, 5 μM) for 10 minutes. Addition of coelenterazine h, a Renilla luciferase substrate, leads to emission of light upon oxidation of coelenterazine h to coelenteramide h, with a peak wavelength around 480 nm. This light energy is then transferred to YFP, provided that the YFP is within an appropriate distance (i.e., 10 nm) and/or orientation, and in turn results in energy emission with a peak wavelength around 530 nm. Subsequent to addition of coelenterazine h, the cells were stimulated with nicotinic acid or MK-0354, and light emission was detected (460-500 nm for Luc and 510-550 nm for YFP) using a Multilabel Reader Mithras LB 940 (Berthold Technologies). The BRET signal was determined as the ratio of the light emitted by YFP and the light emitted by Luc. For dose response curves, different concentrations of ligands were used, and the BRET ratio was monitored at 10 minutes after ligand stimulation. For time kinetics, 10 μM nicotinic acid was added to the cells, and real-time change in BRET was monitored over 15 minutes. The values were corrected by subtracting the background BRET signals detected when Luc-β-arr was expressed alone.

Immunoblotting and immunoprecipitation. Phosphorylated ERK immunoblotting using the antibody anti-phospho-p44/42 MAPK (diluted 1:2,000; Cell Signaling Technology) was carried out as previously described (Wisler et al, Proc. Natl. Acad. Sci. USA 104:16657-16662 (2007)). Total ERK1/2 was detected with anti-MAPK1/2 (diluted 1:3,000; Upstate Biotechnology). Detection β-arrestin1 and β-arrestin2 was performed using rabbit polyclonal antibodies (A1CT and A2CT, respectively) that were generated as previously described (Attramadal et al, J. Biol. Chem. 267:17882-17890 (1992)). Anti-rabbit and anti-mouse secondary antibodies for Western blots were obtained from Amersham Biosciences. For immunoprecipitation, cells were treated with serum-free media with and without nicotinic acid. Cells were washed once with PBS at 4° C., harvested by gentle scraping, pelleted, and resuspended in glycerol lysis buffer including protease and phosphatase inhibitors. Lysates were normalized for equal protein concentrations and immunoprecipitated with A1CT or conjugated M2-beads (Sigma-Aldrich) (Attramadal et al, J. Biol. Chem. 267:17882-17890 (1992)). Immunoprecipitation reactions were incubated at 4° C. for 3 hours, washed 3 times with glycerol lysis buffer, and resuspended in SDS running buffer. Samples were subjected to SDS-PAGE analysis and Western blotting with cPLA₂ antibody (diluted 1:1,000; Cell Signaling Technology), phosphorylated cPLA₂ antibody (diluted 1:1,000; Cell Signaling Technology), M2 antibody (diluted 1:2,000; Sigma-Aldrich), and β-arrestin antibody (diluted 1:1,000; BD Biosciences).

Silencing of gene expression with siRNA. siRNA gene silencing was carried out with previously described siRNAs and methods (Ahn et al, Proc. Natl. Acad. Sci. USA 100:1740-1744 (2003)). Protein silencing of β-arrestin1 and β-arrestin2 was confirmed by immunoblotting. Only experiments with confirmed protein silencing were analyzed.

Animal use and protocols. All animal studies were reviewed and approved by the Duke University Internal Animal Care and Use Committee. Congenic C57BL/6 wild-type mice, β-arrestin1-depleted mice, and β-arrestin2-depleted mice were developed and maintained as previously described (Bohn et al, Science 286:2495-2498 (1999)). Briefly, congenic C57BL/6 wild-type mice, β-arrestin1-deficient, or β-arrestin2-deficient animals were bred, and progeny genotypes were confirmed by PCR and Southern blots. Age- and weight-matched male mice over 12 weeks of age were used in all experiments. Nicotinic acid was resuspended in 5% (2-hydroxypropyl)-β-cyclodextrin in PBS, and the pH was adjusted to 7.4. For FFA assays, mice were food deprived for 8 hours, then treated With 0, 10, 50, or 100 mg/kg nicotinic acid administered by i.p. injection. The animals were euthanized 30 minutes later. Serum was collected and stored at −80° C. Nonesterified FFAs were measured using a Hitachi 911 clinical autoanalyzer, with standards and reagents from Wako USA as previously described (Haqq et al, Contemp. Clin. Trials 26:616-625 (2005)). For mouse cutaneous flushing assays, mice were anesthetized with Nembutal (80 mg/kg) via i.p. injection. After 10 minutes, the mice were placed under an LDPI laser Doppler (PeriScan PIM II; Perimed). The right ear was everted to expose the anterior/ventral surface. The laser Doppler was focused on the central portion of the ventral ear. Data were collected using the repeated data collection mode with a 5-mm×5-mm image size, a 10-second delay, and high-resolution scan. After a 5-minute baseline scan was obtained, 100 mg/kg nicotinic acid was injected in the i.p. space. Readings were continually recorded for 30 minutes. As a control, each animal was subsequently treated with 4 mg/kg PGD₂ dissolved in PBS. For eicosanoid release assays, thioglycollate-elicited peritoneal macrophages were collected as previously described (Misra and Pizzo, Arch. Biochem. Biophys. 379:153-160 (2000), Misra and Pizzo, J. Biol. Chem. 277:4069-4078 (2002)). Cells were pretreated with 0.1 mCi/ml H3-arachidonic acid for 24 hours, then rinsed 5 times to remove unincorporated H3-arachidonic acid (Levine, BMC Cancer. 3:24 (2003)). Macrophages (1×10⁶ cells/well) were stimulated for 10 minutes with 200 μM nicotinic acid, and radioactivity released into the media was measured using a Packard 2700 TR liquid scintillation counter.

Statistics. Significance of differences was determined by 2-way ANOVA with post-hoc Bonferroni tests or 2-tailed Student's paired t tests, using Prism software (version 4; GraphPad). A P value less than 0.05 was considered statistically significant.

### Results

GPR109A has previously been shown to couple to the heterotrimeric G proteins Gᵢ/Gₒ (Richman et al, J. Biol. Chem. 282:18028-18036 (2007), Sakai et al, Arterioscler. Thromb. Vasc. Biol. 21:1783-1789 (2001)). Stimulation of the receptor decreases cAMP, and this response is sensitive to pertussis toxin (Tunaru et al, Nat. Med. 9:352-355 (2003)). To investigate the cellular signaling properties of GPR109A, GPR109A-expressing stable HEK-293 cell lines were established. Based on radioligand binding with nicotinic acid, these cells expressed the receptor at 1,300 mmol/mg total membrane protein (data not shown). After stimulation with nicotinic acid, cAMP decreased, and, as previously reported by others (Tunaru et al, Nat. Med. 9:352-355 (2003)), this response was sensitive to pertussis toxin (FIG. 1A).

Next a determination was made as to whether nicotinic acid—mediated stimulation promotes β-arrestin recruitment to the GPR109A receptor, and whether β-arrestins play a role in GPR109A-mediated signaling. GPR109A-expressing stable cells were transfected with either monomeric yellow fluorescent protein—tagged (mYFP-tagged) β-arrestin1 (referred to herein as β-arrestin1-mYFP) or β-arrestin2-mYFP. In the absence of nicotinic acid, both β-arrestin1 and β-arrestin2 were localized primarily in the cytoplasm (FIG. 1B), with a small amount of β-arrestin2 at the cell membrane. Stimulation with nicotinic acid resulted in robust recruitment of either β-arrestin—mYFP isoform to the cell membrane and a qualitative decrease in cytoplasmic fluorescence (FIG. 1B).

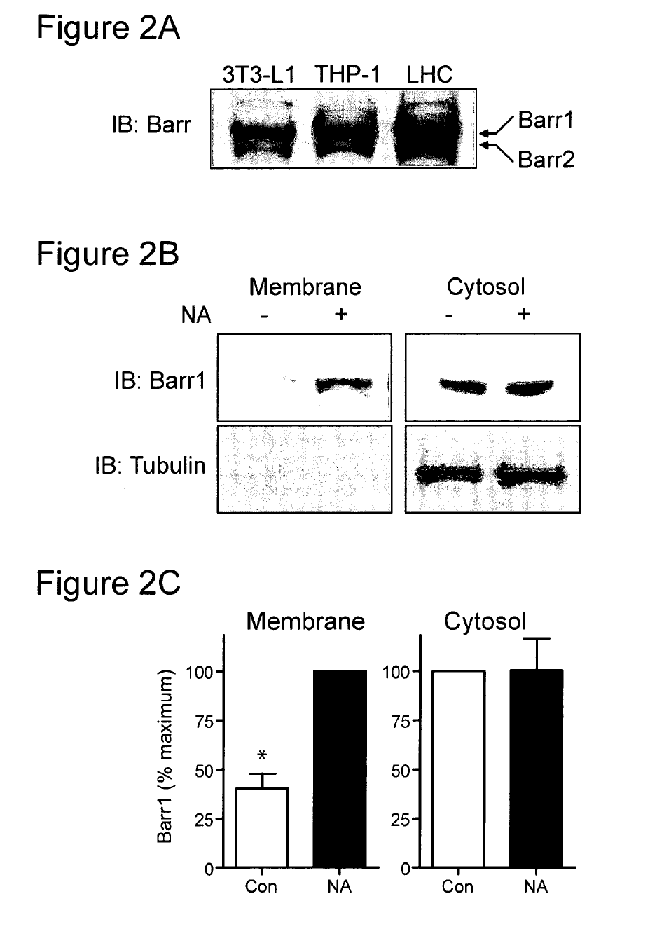

To determine whether β-arrestins are expressed in cells mediating the physiologic response of GPR109A and whether β-arrestin recruitment occurs in response to activation of endogenous receptor, β-arrestin expression was examined in differentiated 3T3-L1 adipocytes, differentiated THP-1 macrophages, and Langerhans cells, and β-arrestin1 membrane recruitment in Langerhans cells was also measured. All 3 cell types expressed both β-arrestin1 and β-arresting (FIG. 2A). In the absence of nicotinic acid, β-arrestin1 remained primarily in the cytosol. Stimulation with nicotinic acid resulted in robust recruitment of β-arrestin1 to the membrane of Langerhans cells (FIGS. 2B and 2C). Nicotinic acid stimulation also led to β-arrestin1 translocation in differentiated THP-1 macrophages (data not shown).

To further characterize the functional interaction of β-arrestin with GPR109A upon nicotinic acid stimulation, a recently described intramolecular bioluminescence resonance energy transfer—based (BRET-based) biosensor was used that detects conformational changes in β-arrestin2 associated with binding an activated 7TMR (Charest et al, EMBO Rep. 6:334-340 (2005)). This biosensor contains bioluminescent Renilla luciferase (Luc) and YFP fused at the N and C termini, respectively, of β-arrestin2 (referred to herein as Luc-β-arr-YFP). Upon recruitment to the receptor, β-arrestin undergoes receptor activation—dependent conformational changes that have been shown to alter the distance and/or orientation of Luc and YFP relative to each other, resulting in an increase in intramolecular BRET efficiency (Charest et al, EMBO Rep. 6:334-340 (2005)). Thus, the Luc-β-arr-YFP biosensor can be used as a reporter for receptor activation as well as for β-arrestin recruitment to the receptor. Stimulation of HEK-293 cells coexpressing GPR109A and the Luc-β-arr-YFP biosensor by nicotinic acid led to an increase in intramolecular BRET ratio in a dose-dependent manner (FIG. 3A). A 50% effective concentration (EC₅₀) of 1.45±0.3×10⁻⁸ M was observed for the conformational change in β-arrestin, which corresponds well with the previously reported K_{d} of the GPR109A receptor (Tunaru et al, Nat. Med. 9:352-355 (2003)) and with the observed IC₅₀ for cAMP production (FIG. 1). Real-time changes in intramolecular BRET ratio upon stimulation of cells with nicotinic acid was also monitored. A time-dependent conformational change in β-arrestin was observed, with a t_{1/2} of maximal BRET increase of 53±5 seconds (FIG. 3B). This time course of conformational change in β-arrestin agrees well with that reported for other class A receptors using this biosensor (Charest et al, EMBO Rep. 6:334-340 (2005)).

Both β-arrestins and G proteins can mediate phosphorylation of ERK after agonist stimulation of Gᵢ/Gₒ-coupled receptors, such as CCR7 and CXCR4 (Cheng et al, J. Biol. Chem. 275:2479-2485 (2000), Kohout et al, J. Biol Chem. 279:23214-23222)). An examination was made as to whether Gᵢ/Gₒ proteins mediated GPR109A-stimulated ERK activation in the stable cell lines in the presence or absence of pertussis toxin. Nicotinic acid did not activate ERK in control HEK-293 cells, which lack GPR109A (data not shown). Phosphorylation of ERK increased after agonist activation with nicotinic acid in GPR109A-expressing cells, and this response was all but eliminated by pertussis toxin, indicating involvement of Gᵢ/Gₒ proteins in this response (FIG. 4A (Tunaru et al, Nat. Med. 9:352-355 (2003)). Next a determination was made as to whether the β-arrestins were also involved in GPR109A-stimulated ERK activation using siRNA targeting either β-arrestin1 or β-arrestin2. Following agonist stimulation, ERK was phosphorylated in control siRNA—transfected cells. In contrast, the response was largely abrogated in cells depleted depleted of β-arrestin1, β-arrestin2, or both (P=0.0023, P=0.0042, and P=0.0017, respectively, versus control, paired 2-tailed Student's t test; FIGS. 4B and 4C). Hence, nicotinic acid stimulation of ERK via GPR109A required both Gᵢ/Gₒ proteins (pertussis toxin sensitive) and β-arrestins, consistent with prior findings for other Gᵢ/Gₒ coupled receptors, including CCR7 and CXCR4 (Cheng et al, J. Biol. Chem. 275:2479-2485 (2000), Kohout et al, J. Biol. Chem. 279:23214-23222)).

ERK phosphorylates and activates cPLA₂, a key enzyme in the production of PGD₂ via production of its precursor arachidonate (Lin et al, Cell 72:269-278)). Because β-arrestin1 is known to interact with other phospholipases, such as phospholipase A₁, in an agonist-dependent manner (Xiao et al, Proc. Natl. Acad. Sci. USA 104:12011-12016 (2007)), an examination was made as to whether β-arrestin interacts with cPLA₂ after stimulation of GPR109A. After stimulation with nicotinic acid, binding of β-arrestin to cPLA₂ and phosphorylated cPLA₂ (the activated form) increased compared with control-treated cells (FIGS. 5A-5D). To determine whether cPLA₂ interacts with β-arrestin1, β-arrestin2, or both, GPR109A-expressing cells were transfected with FLAG-tagged β-arrestin1 or β-arrestin2 and cPLA₂ binding to FLAG-β-arrestin examined. In control-treated cells, low levels of cPLA₂ were bound to both β-arrestin1 and β-arrestin2. After stimulation of GPR109A, binding of cPLA₂ to β-arrestin1 increased, and binding to β-arrestin2 was unchanged (FIGS. 6A and 6C).

To investigate the role of β-arrestin1 in nicotinic acid—stimulated activation of cPLA₂, phosphorylation of cPLA₂ after nicotinic acid stimulation in GPR109A-expressing cells was measured. An increase in phosphorylated cPLA₂ with nicotinic acid was observed, and this response was inhibited by depletion of β-arrestin1 with siRNA (FIGS. 6B and 6D). To determine whether ERK or Gᵢ/Gₒ proteins are involved in nicotinic acid—stimulated phosphorylation of cPLA₂, this response was also measured after pretreatment of cells with either pertussis toxin or the ERK inhibitor PD98059. Both treatments substantially increased the basal level of phosphorylated cPLA₂, and there was no further stimulation by nicotinic acid (FIGS. 6B and 6D). However, because of this large increase in basal cPLA₂ activation after these treatments, it is unclear whether they also actually block nicotinic acid stimulation. Thus, it cannot be firmly concluded that Gᵢ/Gₒ or ERK are involved in this response. The interaction with cPLA₂ was specific for β-arrestin1, and activation of cPLA₂ required β-arrestin1. These data suggest that β-arrestin1 might be required for nicotinic acid—induced cutaneous flushing; moreover, β-arrestins and G proteins may contribute differentially to the therapeutic effects of nicotinic acid on lipids and on the undesired effect of cutaneous flushing.

Nicotinic acid has been shown to decrease serum FFAs and increase cutaneous blood flow in humans and in mice (Pike, J. Clin. Invest. 115:3400-3403 (2005)). Both of these responses require GPR109A, and the decrease in FFAs has also been shown to require Gᵢ/Gₒ proteins (Kather et al, FEBS Lett. 161:149-152 (1983)). This nicotinic acid—induced decrease in serum FFAs has been used as a surrogate for its lipid-lowering effects. The effect of nicotinic acid on serum FFAs was studied in wild-type C57BL/6, β-arrestin1-deficient, and β-arrestin2-deficient mice. Injection of nicotinic acid i.p. to all 3 genotypes produced significant and essentially identical decreases in FFAs (FIG. 7). Statistical analysis by 2-way ANOVA comparing the interaction of dose indicated P<0.0001, and comparing for genotype indicated P=0.92 (wild-type versus β-arrestin1) and P=0.94 (wild-type versus (β-arrestin2). Thus, neither β-arrestin1 nor β-arrestin2 was required for nicotinic acid—induced changes in serum FFAs.

Additionally, changes in cutaneous flushing and eicosanoid release after administration of nicotinic acid were examined by measuring perfusion of the ventral mouse ear using laser Doppler perfusion imaging in vivo and determining cPLA₂ activity in mouse macrophages ex vivo. Injection of nicotinic acid i.p. led to a dramatic increase in perfusion of the ventral ear in both wild-type and β-arresting-deficient mice (FIGS. 8A-8C). Mice deficient in β-arrestin2 showed a trend toward decreased cutaneous flushing that was not statistically significant by 2-way ANOVA, even at the higher 200-mg/kg dose of nicotinic acid (data not shown). Surprisingly,the nicotinic acid—stimulated increase in perfusion was dramatically decreased in the β-arrestin1-deficient mice (FIGS. 8A-8C). Analysis by 2-way ANOVA comparing wild-type with β-arrestin1-deficient mice indicated P=0.0001, and wild-type compared with β-arrestin2-deficient mice indicated P=0.39. As a control, perfusion was measured after injection of PGD₂, the downstream mediator of nicotinic acid—induced vasodilation. Cutaneous flushing increased with PGD₂ in all 3 genotypes, and the response among wild-type, β-arrestin1-deficient, and β-arrestin2-deficient animals was comparable (FIG. 8D). These data clearly demonstrate that β-arrestin1 participates in mediating nicotinic acid—induced cutaneous flushing and that the defect in cutaneous flushing occurs upstream of the actions of prostaglandin on cutaneous blood vessels (i.e., upstream of prostaglandin release).

To further investigate the mechanism by which β-arrestin1 mediates the cutaneous flushing response, peritoneal macrophages were harvested from wild-type, β-arrestin1-deficient, and β-arrestin2-deficient mice and cPLA₂ activity in these cells after stimulation of GPR109A was measured. Macrophages were pretreated with H³-arachidonic acid for 24 hours to allow incorporation into membrane lipids. Release of radiolabeled eicosanoids, a measure of cPLA₂ activity, increased after stimulation with nicotinic acid in wild-type and β-arrestin2-deficient macrophages, and this response was significantly reduced in β-arrestin1-deficient cells (FIG. 8E). A nonsignificant trend toward diminished cutaneous flushing was observed in β-arrestin2-deficient mice; hence, it is speculated that β-arrestin2 could also play some role in this response and may account for part of the residual eicosanoid production in β-arrestin1-deficient macrophages. While the possibility that defective nicotinic acid—induced cutaneous flushing in β-arrestin1-deficient mice involves additional mechanisms cannot be excluded, defective cPLA₂ activity in immune cells markedly limited cutaneous flushing in these animals. Taken together, these findings demonstrate that the adverse side effect of cutaneous flushing associated with the administration of nicotinic acid is mediated by β-arrestin1. In contrast, the effects on serum FFAs are mediated by β-arrestin-independent—Gᵢ/Gₒ protein dependent—mechanisms, which have previously been shown to be pertussis toxin sensitive (Kather et al, FEBS Lett. 161:149-152 (1983)).

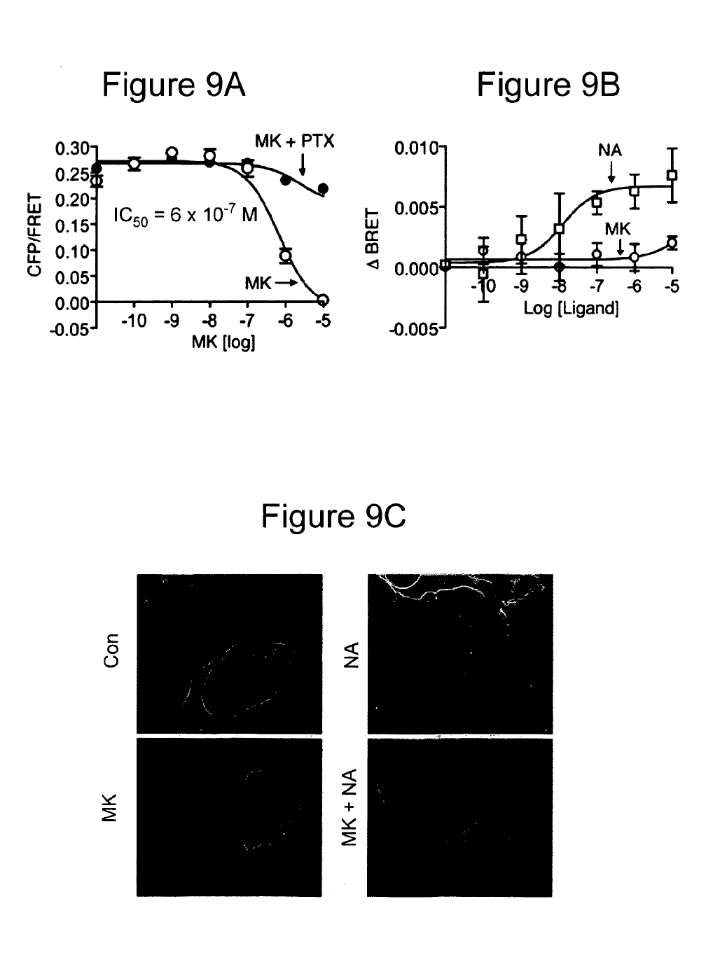

Recently developed GPR109A agonists, such as MK-0354, decrease serum FFAs, but do not induce cutaneous flushing (Sakai et al, Arterioscler. Thromb. Vasc. Biol. 21:1783-1789 (2001), Lai et al, J. Clin. Lipidol. 2:375-383 (2008), Semple et al, J. Mol. Chem. 51:5101-5108 (2008)). It was hypothesized that biased signaling toward Gᵢ/Gₒ proteins might be the mechanism for such biased or selective pharmacology. To test this hypothesis, G protein signaling and β-arrestin recruitment were measured after agonist activation of GPR109A-expressing HEK-293 cells using the agonist MK-0354. As previously demonstrated (Semple et al, J. Mol. Chem. 51:5101-5108 (2008)), stimulation of the receptor with MK-0354 decreased cAMP, and this response was sensitive to pertussis toxin (FIG. 9A). However, stimulation with MK-0354 failed to induce a conformational change in the Luc-β-arr-YFP biosensor as measured by BRET (FIG. 9B). Moreover, MK-0354 failed to induce recruitment of β-arrestin1-mYFP to the cell membrane and inhibited nicotinic acid—induced recruitment (FIG. 9C). Hence, a nonflushing agonist of GPR109A activated G protein signaling, but failed to stimulate recruitment of β-arrestin, perhaps explaining its selective pharmacology. These findings have 2 possible explanations: that MK-0354 is a G protein-biased agonist, or that MK-0354 is a weak partial agonist. Both of these explanations may be consistent with this compound's reported efficacy for FFA lowering in the absence of cutaneous flushing. This selective effect could be achieved by either a biased ligand that engages G protein coupling but not β-arrestin coupling, or by a partial agonist that weakly engages both G protein and β-arrestin coupling and gains selectivity for the G protein response through downstream amplification, which is commonly seen for G protein-coupled responses.

In summary, nicotinic acid inhibits lipolysis in adipocytes, decreasing serum FFAs and triglycerides, and this effect is mediated by GPR109A (Tunaru et al, Nat. Med. 9:352-355 (2003)). In addition to the triglyceride-lowering effect, nicotinic acid also improves a number of other cardiovascular risk factors. Specifically, nicotinic acid is the most effective HDL-raising therapy currently known, and has also been shown to lower both VLDL and LDL (Pike, J. Clin. Invest. 115:3400-3403 (2005)). While the FFA- and triglyceride-lowering effects of nicotinic acid are clearly mediated by GPR109A, it is not yet known whether its beneficial effects on HDL, VLDL, and LDL are also mediated by this receptor (Guyton, Curr. Opin. Lipidol. 18:415-420 (2007)). GPR109A receptors are found primarily in adipose tissue, spleen, adrenal glands, and lungs, and are all but absent from the liver and intestines, which are the main sites of HDL synthesis and metabolism (Tunaru et al, Nat. Med. 9:352-355 (2003), Wise et al, J. Biol. Chem. 278:9869-9874 (2003)). Thus, there may be additional mechanisms of action for the beneficial effects of nicotinic acid on lipoprotein profiles, mediated through sites other than GPR109A. For example, nicotinic acid at high concentrations has been shown to directly inhibit hepatic diacylglycerol acetyltransferase (Ganji et al, J. Nutr. Biochem. 14:298-305 (2003), Jin et al, Arterioscler. Thromb. Vasc. Biol. 19:1051-1059 (1999)), thus inhibiting hepatic triglyceride synthesis, which increases apoB degradation and consequently decreases VLDL and LDL production and secretion.

Nicotinic acid has also been shown, via an as-yet-unidentified mechanism, to inhibit the uptake and removal of HDL by the liver, resulting in increased circulating HDL levels (Jin et al, Arterioscler. Thromb. Vasc. Biol. 17:2020-2028 (1997)). Thus, multiple sites of action may be involved in the pleiotropic actions of nicotinic acid on lipoproteins beyond the clear effect on FFAs and triglycerides mediated through GPR109A. However, GPR109A clearly mediates the prominent side effect of cutaneous flushing (Benyo et al, J. Clin. Invest. 115:3634-3640 (2005)), and does so in a β-arrestin1-dependent fashion. This is in contrast to the desired therapeutic effects on FFAs that are not mediated via β-arrestins.

It has been shown previously for the AT1 angiotensin receptor, parathyroid hormone receptor, and β₂-adrenergic receptor that it is possible to selectively induce either G protein- or β-arrestin-biased signaling with specific ligands (Drake et al, J. Biol. Chem. 283:5669-5676 (2008), Gesty-Palmer et al, J. Biol. Chem. 281:10856-10864 (2006), Wei et al, Proc. Natl. Acad. Sci. USA 100:10782-10787 (2003), Wisler et al, Proc. Natl. Acad. Sci. USA 104:16657-16662 (2007)). Such molecules entrain subsets of receptor signaling pathways without activating all of a receptor's possible downstream effectors. This idea of biased ligands departs from the traditional view of receptor ligands as full agonists, partial agonists, inverse agonists, or antagonists and opens up a much more nuanced framework in which receptor ligands might act independently to activate either G proteins or β-arrestins (Kenakin, Mol. Pharmacol. 72:1393-1401 (2007)). As potential therapeutic agents, such ligands could specifically target therapeutic effectors while avoiding those signaling pathways associated with particular side effects. Indeed, recent studies using novel GPR109A agonists that decrease serum FFAs in mice and humans without inducing cutaneous flushing demonstrated divergent signaling pathways downstream of GPR109A activation (Richman et al, J. Biol. Chem. 282:18028-18036 (2007), Lai et al, J. Clin. Lipidol. 2:375-383 (2008), Semple et al, J. Med. Chem. 51:5101-5108)). Specifically, compounds such as MK-0354 activate G proteins, but fail to induce ERK activation and internalization of the receptor. These observations suggest that MK-0354 preferentially activates G proteins over β-arrestins, further supporting the notion that it is possible to specifically target the beneficial effects of GPR109A signaling while avoiding signaling pathways that mediate cutaneous flushing.

While most patients taking nicotinic acid experience cutaneous flushing, some are able to tolerate this side effect, mostly because tolerance to cutaneous flushing sometimes occurs with prolonged use of the medication. The mechanism of tolerance to nicotinic acid—induced cutaneous flushing is not understood, and such information may also lead to improvements in nicotinic acid—based therapies. The identification of GPR109A as a receptor for nicotinic acid (Benyo et al, J. Clin. Invest. 115:3634-3640 (2005)), coupled with the finding that β-arrestin proteins are recruited after activation of this receptor, leads to the speculation that β-arrestins may also internalize GPR109A and desensitize GPR109A-mediated signaling. Hence, these proteins may also play a role in tolerance to nicotinic acid—induced cutaneous flushing. However, since β-arrestin-mediated receptor internalization would also be predicted to desensitize G protein signaling, and tolerance to the beneficial effects of nicotinic acid on serum lipids does not occur, the role of β-arrestins in desensitization of GPR109A-mediated signaling is likely to be complicated.

In conclusion, the adverse side effect of cutaneous flushing associated with nicotinic acid was mediated by β-arrestin1, while the effects on lowering serum FFAs were not. Thus, agents that possess the FFA- and triglyceride-altering attributes of nicotinic acid but do not activate β-arrestin recruitment to GPR109A can be predicted to lack the side effect of cutaneous flushing. Such biased ligands would provide a significant therapeutic advantage over currently available medications used to treat hypertriglyceridemia and potentially other dyslipidemias. Moreover, screening for GPR109A agonists that stimulate activation of G proteins but not β-arrestin1 provides a strategy for their identification. These findings provide a striking example of how desired therapeutic and unwanted side effects of GPCR-targeted drugs can be dissociated with respect to molecular signaling pathways through G proteins and β-arrestins.

### EXAMPLE 2

The flushing effects of nicotinic acid are subject to the development of tolerance, over weeks to months, while the effects on lipids remain intact. Relatively high doses (1000-2000 mg/day) of nicotinic acid are required to realize the positive lipid effects, whereas the unwanted cutaneous vasodilation or flush can occur with even lower doses (100-200 mg/day). Further, dose escalation upon tolerance is often used as a therapeutic strategy in an attempt to achieve the lipid effects while minimizing the flushing side effect over time. However, with each dose escalation, the flushing often returns.

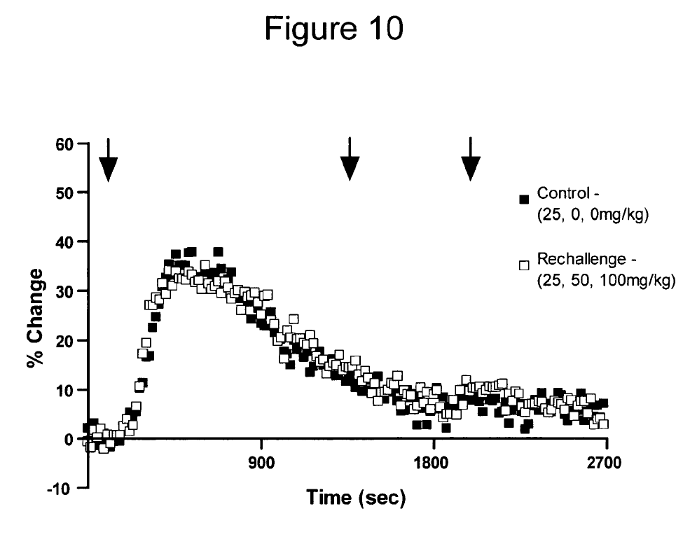

A study was undertaken to determine the effects of acute nicotinic acid re-challenge and dose escalation on ventral ear perfusion in age-matched wild type mice. Treatment with either a single injection of nicotinic acid (25 mg/kg) or an initial injection of nicotinic acid (25 mg/kg) followed by increased doses of nicotinic acid (50 & 100 mg/kg) resulted in a similar flushing profile (FIG. 10). These data clearly show that there is rapid tachyphylaxis to the cutaneous vasodilation/flushing associated with a moderate dose of nicotinic acid, and this tachyphylaxis is not overcome by increasing the doses of nicotinic acid. Moreover, mice still respond to exogenous prostaglandin D2 after receiving nicotinic acid, identifying the site of the signaling defect upstream of prostaglandin D2 receptor activation.

All documents and other information sources cited above are hereby incorporated in their entirety by reference.

What is claimed is:

1. A method of identifying a biased ligand for the G protein coupled receptor GPR109A (GPR109A) comprising:
i) determining the effect of a test compound on GPR109A-mediated G protein activity, and
ii) determining the effect of said test compound on a GPR109A-mediated β-arrestin function,
wherein a test compound that has a greater positive effect on said GPR109A-mediated G-protein activity than on said GPR109A-mediated β-arrestin function, relative to a reference agonist for both said GPR109A-mediated G-protein activity and said GPR109A-mediated β-arrestin function, is a biased ligand for GPR109A.

2. The method according to claim 1 wherein said GPR109A is present in a eukaryotic cell.

3. The method according to claim 1 wherein step (i) is effected by measuring the level of calcium, cyclic adenosine monophosphate (cAMP), diacylglycerol or inositol triphosphate in the presence and absence of said test compound.

4. The method according to claim 1 wherein step (i) is effected by measuring phosphatidyl inositol turnover, GTP-γ-S loading, adenylate cyclase activity or GTP hydrolyis in the presence and absence of said test compound.

5. The method according to claim 1 wherein step (ii) is effected by measuring β-arrestin or GRK recruitment to, or internalization or GRK-mediated phosphorylation of, said GPR109A in the presence and absence of said test compound.

6. The method according to claim 5 wherein step (ii) is effected by measuring said β-arrestin recruitment to said GPR109A by assaying the physical interaction between β-arrestin and said GPR109A.

7. The method according to claim 5 wherein said β-arrestin recruitment is measured by resonance energy transfer, bimolecular fluorescence, enzyme complementation, visual translocation, co-immunoprecipitation, membrane association, or interaction of β-arrestin with a naturally occurring binding partner.

8. The method according to claim 7 wherein said GPR109A is present in a eukaryotic cell and said β-arrestin recruitment is measured by resonance energy transfer.

9. The method according to claim 8 wherein said cell co-expresses said GPR109A fused to a first fluorescent protein (GPR109A fusion protein) and β-arrestin fused to a second fluorescent protein (β-arrestin fusion protein), wherein said first and second fluorescent proteins undergo fluorescence resonance energy transfer (FRET) upon interaction of said β-arrestin fusion protein with said GPR109A fusion protein, and
wherein said β-arrestin recruitment is determined by measuring the FRET increase in the presence of said test compound.

10. The method according to claim 9 wherein said first fluorescent protein is a monomeric cyan variant of Green Fluorescent Protein and said second fluorescent protein is a yellow variant of Green Fluorescent Protein.

11. The method according to claim 1 wherein step (i) effected by measuring the level of cAMP cell in the presence and absence of said test compound and step (ii) is effected by measuring the β-arrestin recruitment to said GPR109A in the presence and absence of said test compound.

12. A method of identifying a candidate therapeutic that reduces triglyceride levels in a patient comprising:
i) determining the effect of a test compound on G-protein activity mediated by GPR109A, and
ii) determining the effect of said test compound on a β-arrestin function mediated by GPR109A,
wherein a test compound that has a greater positive effect on said G-protein activity mediated by GPR109A than on said β-arrestin function mediated by GPR109A, relative to a reference agonist for both said G-protein activity mediated by GPR109A and said β-arrestin function mediated by GPR109A, is said candidate therapeutic.

13. The method according to claim 12 wherein said method is a method of identifying a candidate therapeutic that reduces triglyceride levels and increases high density lipoprotein levels in said patient.

14. The method according to claim 13 wherein said method is a method of identifying a candidate therapeutic that reduces triglyceride levels, increases high density lipoprotein levels and reduces low density or very low density lipoprotein levels in said patient.

15. A method of reducing triglyceride levels comprising administering to a patient in need thereof a compound that is an agonist of G-protein activity mediated by GPR109A and that has a greater positive effect on G-protein activity mediated by GPR109A than on β-arrestin function mediated by GPR109A, relative to a reference agonist for both said G-protein activity mediated by GPR109A and said β-arrestin function mediated by GPR109A, wherein said compound is administered in an amount such that said reduction is effected.

16. The method according to claim 15 wherein said method is a method of reducing triglyceride levels and increasing high density lipoprotein levels in said patient.

17. The method according to claim 16 wherein said method is a method of reducing triglyceride levels, increasing high density lipoprotein levels and reducing low density or very low density lipoprotein levels in said patient.

18. A method of reducing triglyceride levels in a subject in need thereof comprising administering to said subject an initial low to moderate dose of a biased ligand for GPR109A identifiable by the method according to claim 1 closely followed by a larger therapeutically effective dose of said biased ligand, thereby reducing said triglyceride levels with minimal flushing.

19. A method of reducing triglyceride levels in a subject in need thereof comprising: i) administering to said subject 50-300 mgs of nicotinic acid, ii) about 30 minutes after step (i), administering to said subject about 1000-2000 mgs of nicotinic acid, thereby reducing said triglyceride levels with minimal flushing.
